(19) United States
(12) Patent Application Publication
(10) Pub. No.: US 2026/0207650 A1
(43) Pub. Date: Jul. 23, 2026

(54) PROLIFERATION INHIBITOR OF BREAST CANCER CELLS AND THERAPEUTIC AGENT FOR BREAST CANCER

(71) Applicants: Medical Corporation Sugiyama General Medicine, Nagoya-shi (JP); Elysiun Science Co., Ltd., Moriyama-shi (JP)

(72) Inventors: Satoru SUGIYAMA, Nagoya-shi (JP); Kiyoaki KAWAHARA, Moriyama-shi (JP)

(73) Assignees: Medical Corporation Sugiyama General Medicine, Nagoya-shi (JP); Elysiun Science Co., Ltd., Moriyama-shi (JP)

(21) Appl. No.: 19/219,867

(22) Filed: May 27, 2025

(30) Foreign Application Priority Data
Jan. 23, 2025 (JP) 2025-009547

(51) Int. Cl. A61K 31/7105; A61P 35/00

(52) U.S. Cl. CPC A61K 31/7105; A61P 35/00

(57) ABSTRACT

A proliferation inhibitor of breast cancer cells and a therapeutic agent for breast cancer include an RNA molecule of a nucleotide sequence set forth in SEQ ID NO: 1, wherein the RNA molecule includes a 5′ cap. SEQ ID NO: 1 represents the base sequence of zapomeran, which is an mRNA vaccine for spike glycoprotein binding to an ACE2 receptor present on the surface of human cells. Note that the RNA molecule is preferably encapsulated in a lipid nanoparticle (LNP).

### REFERENCE TO ELECTRONIC SEQUENCE LISTING

The application contains a Sequence Listing which has been submitted electronically in .XML format and is hereby incorporated by reference in its entirety. Said .XML copy, created on May 9, 2025, is named “PA0514. xml” and is 13,459 bytes in size. The sequence listing contained in this .XML file is part of the specification and is hereby incorporated by reference herein in its entirety.

### BACKGROUND OF THE INVENTION

### (1) Field of the Invention

The present invention relates to a proliferation inhibitor of breast cancer cells and a therapeutic agent for breast cancer.

### (2) Description of Related Art

COVID-19 is an infectious disease caused by a novel coronavirus SARS-COV-2 that appeared in 2019, and vaccine development has been made from an early stage as a countermeasure for infection spread (JP 2024-15475 A and the like). A coronavirus is an enveloped virus having a positive-sense single-stranded RNA encoding four structural proteins as a viral genome. Among these four structural proteins, a spike glycoprotein that binds to an ACE2 receptor present on the surface of a human cell has been targeted for vaccine development.

An mRNA vaccine thus developed is zapomeran. Zapomeran is a self-replicating mRNA encoding Venezuelan equine encephalitis virus RNA replicase (nsP1, nsP2, nsP3, nsP4) and the full length of a spike protein analog of SARS-COV-2 (D614G, R682G, R683S, R685S, K986P, V987P), and is a single-stranded RNA consisting of 11861 nucleotide residues including a 5′ cap structure, a subgenomic promoter sequence, and a polyA sequence (see FIGS. 1 and 2 and SEQ ID NO: 1).

Meanwhile, in recent years, drug repositioning (DR) has attracted attention as a technique for developing a therapeutic agent for a disease. Drug repositioning (DR) is a development technique in which an existing drug or pharmaceutical product/compound under development or discontinued is utilized and diverted as a therapeutic agent for a disease different from the original assumption.

In development of a new drug by drug repositioning (DR), there is an advantage that clinical trials can proceed quickly because basic safety has already been confirmed. In addition, it is not necessary to develop a new pharmaceutical product from scratch, and thus there is also an advantage that research and development costs can be suppressed.

However, development of an anticancer agent by drug repositioning (DR) on an mRNA encoding the spike protein analog of SARS-COV-2 is not known.

### SUMMARY OF THE INVENTION

The present invention is directed to providing a proliferation inhibitor of breast cancer cells and a therapeutic agent for breast cancer to which an mRNA vaccine encoding a spike protein analog of the novel coronavirus SARS-COV-2 is diverted.

The present inventors have found that among mRNA vaccines encoding spike protein analogs of SARS-COV-2, zapomeran has a remarkable proliferation inhibitory effect on human breast cancer cells, and have completed the present invention.

That is, a proliferation inhibitor of breast cancer cells of the present invention includes an RNA molecule of a nucleotide sequence set forth in SEQ ID NO: 1, wherein the RNA molecule includes a 5′ cap.

The term “5′ cap” refers to a structure found on the 5′ end of an mRNA molecule and includes a guanosine nucleotide linked to mRNA via a 5′ to 5′ triphosphate linkage. The present inventors have confirmed that when the 5′ cap has the following chemical structure, a remarkable proliferation inhibitory effect is reliably exerted on human breast cancer cells.

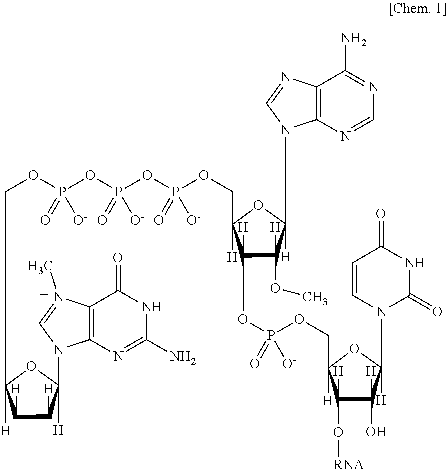

The RNA molecule is preferably encapsulated in a lipid nanoparticle (LNP). The lipid nanoparticle (LNP) has, for example, a form enclosed in an LNP or a form associated with an LNP. The LNP may include any lipid to which one or more nucleic acid molecules bind or which is capable of forming a particle in which one or more nucleic acid molecules are encapsulated. The LNP includes a cationic lipid, a neutral lipid, a steroid, a polymer-conjugated lipid, and an RNA.

A therapeutic agent for breast cancer cells of the present invention includes an RNA molecule of a nucleotide sequence set forth in SEQ ID NO: 1, wherein the RNA molecule includes a 5′ cap.

The present inventors have confirmed that when the 5′ cap has the following chemical structure, a remarkable proliferation inhibitory effect is reliably exerted on human breast cancer cells.

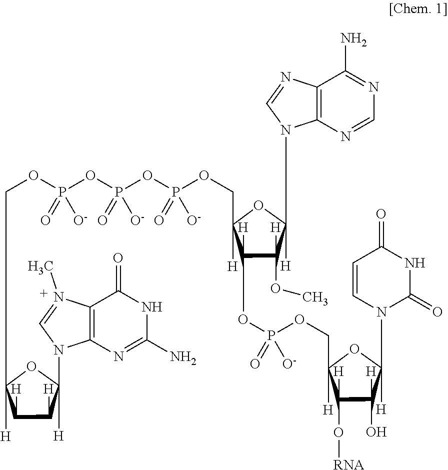

The RNA molecule is preferably encapsulated in a lipid nanoparticle (LNP). The lipid nanoparticle (LNP) has, for example, a form enclosed in an LNP or a form associated with an LNP. The LNP may include any lipid to which one or more nucleic acid molecules bind or which is capable of forming a particle in which one or more nucleic acid molecules are encapsulated. The LNP includes a cationic lipid, a neutral lipid, a steroid, a polymer-conjugated lipid, and an RNA.

### BRIEF DESCRIPTION OF THE DRAWINGS

FIG. 1 is a sequence listing of zapomeran (SEQ ID NO: 1);

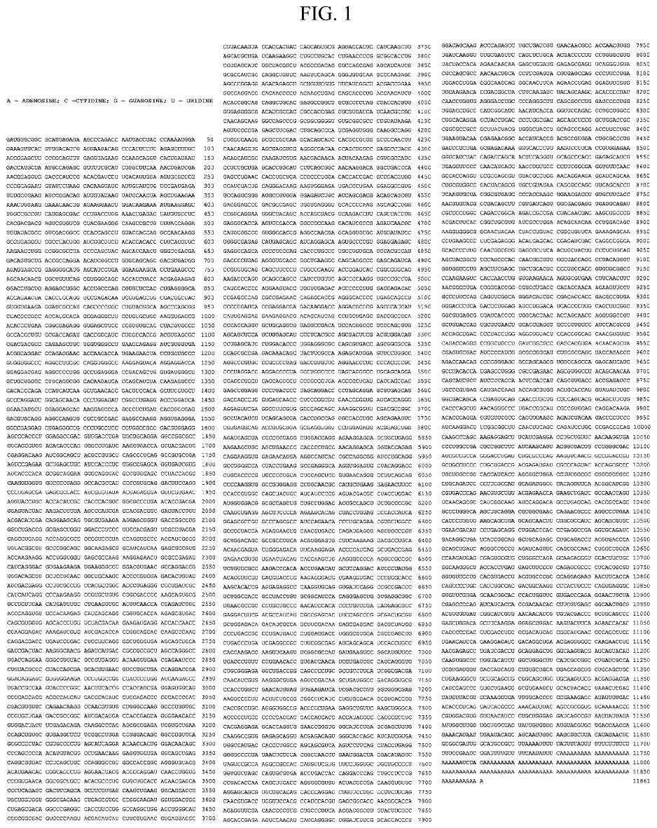

FIG. 2 is a chemical structural formula of a 5′ cap structural portion of zapomeran;

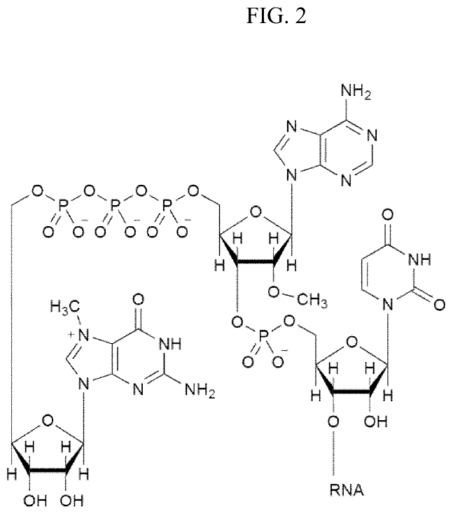

FIG. 3 is a graph showing BT-474 cell viability of a test substance (iABC2024K)—added group and a saline-added group in plate.1;

FIG. 4 is a graph showing BT-474 cell viability of a test substance (iABC2024K)—added group and a saline-added group in plate. 2; and

FIG. 5 is a graph showing BT-474 cell viability of a test substance (iABC2024K)—added group and a saline-added group in plate. 3.

### DETAILED DESCRIPTION OF PREFERRED EMBODIMENTS

Hereinafter, the proliferation inhibitor of breast cancer cells and the therapeutic agent for breast cancer of the present invention will be described in more detail with reference to Examples.

### EXAMPLES

An evaluation test for the cell proliferation inhibitory effect of iABC2024K on human breast cancer cell line BT-474 cells was performed. iABC2024K is Kostaive Intramuscular Injection manufactured by Meiji Seika Pharma Co., Ltd., and contains zapomeran developed as an mRNA vaccine as an active ingredient.

### <Materials and Methods Used in Evaluation Test>

### (Test Substance)

### i ABC2024K

Container: vial
Properties: white dry formulation
Storage conditions: −20° C., stored in an allowable range of ±5.0° C. (−25.0 to −15.0° C.), light-shielding

### Test Substance Stock Solution

For iABC2024K, the test substance stock solution was prepared by returning the vial to room temperature, adding a total of 9 mL of saline to the dry formulation in the vial in 3 portions, and dissolving the mixture.

### Preparation of Dilution Series of Test Substance

Each test substance stock solution was repeatedly diluted 2 times with saline as a medium to prepare diluted solutions of 2 times (relative concentration: 0.5), 4 times (relative concentration: 0.25), 8 times (relative concentration: 0.125), and 16 times (relative concentration: 0.0625) the test substance stock solution (relative concentration: 1). The diluted solutions were prepared before use.

### <Reagents and Materials>

The reagents and materials used are shown in Table 1.

TABLE 1

| Reagents and | | | Storage |
|---|---|---|---|
| materials | Manufacturer | Catalog No. | temperature |
| 100 mm × 20 mm | Coming | 353003 | Room |
| cell culture dish | | | temperature |
| Cell culture 96- | Coming | 353072 | Room |
| well multiwell | | | temperature |
| plate flat bottom | | | |
| 1-L storage | Coming | 430518 | Room |
| bottle | | | temperature |
| 0.22-μm bottle | Coming | 430513 | Room |
| top filter | | | temperature |
| Hybri-Care Medium | ATCC | 46-X | 2.0 to |
| | | | 8.0° C. |
| Sodium | FUJIFILM | 197-01302 | Room |
| bicarbonate | Wako Pure | | temperature |
| | Chemical | | |
| Fetal Bovine | Cytiva | SH30910.03 | −40.0 to |
| Serum | | | −20.0° C. |
| Penicillin- | Thermo | 15070063 | −40.0 to |
| Streptomycin | Fisher | | −20.0° C. |
| | Scientific | | |
| DPBS, no calcium, | Thermo | 14190144 | Room |
| no magnesium | Fisher | | temperature |
| | Scientific | | |
| TrypLE Select | Thermo | 12563011 | Room |
| Enzyme (1x), no | Fisher | | temperature |
| phenol red | Scientific | | |
| 0.4% trypan blue | Thermo | 15250061 | Room |
| | Fisher | | temperature |
| | Scientific | | |
| Cell calculation | funakoshi | 521-10 | Room |
| board | | | temperature |
| STEM-CELLBANKER | TaKaRa | 11924 | 2.0 to |
| GMP grade | | | 8.0° C. |
| Cell Counting | FUJIFILM | 343-07623 | 2.0 to |
| Kit-8 | Wako Pure | | 8.0° C. |
| | Chemical | | |

### <Devices and Instruments>

The devices and instruments used are shown in Table 2.

TABLE 2

| Name | Manufacturer | Model | SOP No. |
|---|---|---|---|
| Liquid nitrogen | MINNESOTA | MVE XC | ME301 |
| storage container | VALLEY ENGR | 34/18 | |
| Electronic balance | A & D Company | GR-202 | ME101 |
| for reagent | | | |
| measurement | | | |
| Ultrapure water | Merck | Milli-Q ® IQ | ME201 |
| production system | Millipore | 7003 | |
| pH meter | HORIBA | F-52 | ME403 |
| Cold storage | PHC | MPR- | ME309 |
| chamber for | | N450FSH-PJ | ME310 |
| refrigerator | | | |
| medicine | | | |
| Ultra-low | PHC | MDF- | ME303 |
| temperature | | DC700VX-PJ | |
| freezer | | | |
| Digital | Eppendorf | Reference | ME421 |
| micropipette | | 4910, | |
| | | Reference 2 | |
| Multichannel | Thermo Fisher | F1 Clip Tip | ME406 |
| pipette | Scientific | 12ch 10- | |
| | | 100 μL | |
| Clean bench | TOSC Japan | NS-13BS | ME024 |
| Constant | AS ONE | TR-S | ME207 |
| temperature water | | | |
| bath | | | |
| Cooling centrifuge | KUBOTA | 5910 | ME409 |
| Carbon dioxide gas | PHC | MCO- | ME261 |
| incubator | | 170AICUVD- | |
| | | PJ | |
| Inverted phase | Nikon | Nikon | ME353 |
| contrast | | TE2000-S | |
| microscope | | | |
| Microplate reader | Molecular | SpectraMax | ME436 |
| | Devices | iD3 | |

### <Cell Culture>

### Test Culture

### Name: Human Breast Cancer Cell Line BT-474 Cells (Hereinafter, Referred to as BT-474)·

### Complete Medium

Hybri-Care Medium, in which inactivated Fetal Bovine Serum (hereinafter, FBS) was added so as to have a final concentration of about 10%, and Penicillin-Streptomycin was added so as to have a final concentration of about 50 units/mL, Penicillin G, and 50 μg/mL of Streptomycin, was used as a complete medium. The medium was refrigerated at 2.0 to 8.0° C.

### Thawing of BT-474 Cell Frozen Stock

1) The complete medium was warmed in the constant temperature water bath set at 37° C.
2) Frozen stock of cells was thawed in the constant temperature water bath set at 37° C.
3) Thawed cells were dispersed in 9 mL of the complete medium.
4) 200 g of the dispersion was centrifuged at 20° C. for 5 minutes, and the whole amount of the supernatant was removed.
5) Cells corresponding to one cryotube were dispersed in 10 mL of the complete medium and seeded on a 100 mm×20 mm cell culture dish.
6) Immediately after 5), the cells were observed under a microscope. The cell density was appropriate, and thus no operation such as thinning out the cells was performed.
7) The cells were cultured in the carbon dioxide gas incubator set at 37° C. and 5% CO₂.
8) The whole amount of the medium was changed at a frequency of 2 or 3 times per week until passage.

### Passage of BT-474 Cells

Hereinafter, the method at the time of passage to one 100 mm×20 mm cell culture dish will be described. When the size of the culture vessel or the number of culture vessels was changed, the liquid amounts of the medium and reagents used were proportionally increased or decreased.
1) The complete medium was warmed in the constant temperature water bath set at 37° C.
2) The cell density and state of the cells before passage were observed with a microscope.
3) The whole amount of the old medium was removed from the culture vessel, and the cells were washed with 10 mL of DPBS, no calcium, and no magnesium (hereinafter, referred to as D-PBS(−)).
4) The whole amount of D-PBS(−) was removed, and 2 mL of TrypLE Select Enzyme was added to spread to the entire cells, and then the cells were incubated at 37° C. for approximately 5 minutes until they were peeled off.
5) The culture vessel was patted to peel off the cells from the vessel bottom.
6) 5 mL of the complete medium was added thereto, and the cells were suspended by pipetting and then collected in a 15-mL tube.
7) 200 g of the suspension was centrifuged at 20° C. for 5 minutes, and the whole amount of the supernatant was removed.
8) The cells were suspended in 5 mL of the complete medium and the number of cells was counted.
9) An amount of ½ to ⅓ of the cell suspension of 8) was seeded on one 100 mm×20 mm cell culture dish, and the complete medium was added so that the amount of the culture solution was 10 mL.
10) The cells were cultured in a carbon dioxide gas incubator set at 37° C. and 5% CO₂. When there is time until the next passage, the whole amount of the medium was changed at a frequency of 2 or 3 times per week.

### Cryopreservation of Cells

Frozen stock was made from cells remaining at passage and stored in liquid nitrogen until the end of the test.
1) 200 g of cells remaining after passage were centrifuged at 20° C. for 5 minutes, and the whole amount of the supernatant was removed.
2) 2 mL of STEM-CELLBANKER GMP grade was added to the cell pellet and well suspended.
3) The cell suspension was dispensed into two cryotubes at 1 mL/tube and cryopreserved at −80° C. After freezing at −80° C. for one or more days, the cells were transferred to a liquid nitrogen storage container.

The remainder of the frozen stock of cells was discarded as an infectious waste after the end of the test.

### <Drug Sensitivity Test of BT-474 Cells>

The proliferation inhibitory effect of the test substance on BT-474 cells was evaluated.
1) BT-474 cells which were suspended in the complete medium so as to be 5.56×10⁴ cells/mL were seeded on three 96-well plates (plate.1, plate.2, and plate. 3) at 180 μL/well (1.00×10⁴ cells/well) in the arrangement shown in Table 3. In all the 96-well plates, the complete medium for preventing drying was only dispensed at 200 μL/well to wells in rows A and H and columns 1 and 12 in which the cell suspension was not seeded, and the wells were not used for analysis. On the other hand, in the wells in columns 2 to 11 of row G, the complete medium was dispensed at 180 μL/well, and the wells were used as blank wells at the time of analysis.

TABLE 3
Arrangement diagram of cell seeding number or medium
dispensing (plate.1, plate.2, and plate.3)

| | 1 | 2 | 3 | 4 | 5 | 6 | 7 | 8 | 9 | 10 | 11 | 12 |
|---|---|---|---|---|---|---|---|---|---|---|---|---|
| A | Me- | Me- | Me- | Me- | Me- | Me- | Me- | Me- | Me- | Me- | Me- | Me- |
| | dium | dium | dium | dium | dium | dium | dium | dium | dium | dium | dium | dium |
| B | Me- | Seeding 5.56 × 10⁴ cells/mL at 180 μL/well | | | | | | | | | | Me- |
| | dium | (1.00 × 10⁴ cells/well) | | | | | | | | | | dium |
| C | Me- | | | | | | | | | | | Me- |
| | dium | | | | | | | | | | | dium |
| D | Me- | | | | | | | | | | | Me- |
| | dium | | | | | | | | | | | dium |
| E | Me- | | | | | | | | | | | Me- |
| | dium | | | | | | | | | | | dium |
| F | Me- | | | | | | | | | | | Me- |
| | dium | | | | | | | | | | | dium |
| G | Me- | Medium (blank well) | | | | | | | | | | Me- |
| | dium | | | | | | | | | | | dium |
| H | Me- | Me- | Me- | Me- | Me- | Me- | Me- | Me- | Me- | Me- | Me- | Me- |
| | dium | dium | dium | dium | dium | dium | dium | dium | dium | dium | dium | dium |

2) Six 96-well plates on which cells were seeded were subjected to culture in the carbon dioxide gas incubator set at 37° C. and 5% CO₂ until the next day.
3) The dilution series of each test substance prepared before use and saline were added to the 96-well plates after culture of 2) at 20 μL/well according to Table 5.
4) plate.1, plate.2, and plate. 3 were subjected to culture for 3 days, 5 days, and 7 days, respectively, in the carbon dioxide gas incubator set at 37° C. and 5% CO₂.
5) Cell Counting Kit-8 was added to the 96-well plates after completion of the culture of 4) at 20 μL/well, and the plates were subjected to incubation in the carbon dioxide incubator set at 37° C. and 5% CO₂.
6) Absorbance at 450 nm (reference wavelength 600 nm) was measured on the microplate reader about 1 hour, about 2 hours, and about 4 hours after the addition of Cell Counting Kit-8 in 5).
7) The cell viability was calculated by the following equation. The cell viability was graphed for different drug concentrations.

$$[ Math . 1 ]$$

$$Cell viability ( \% ) = [ ( As - Ab ) / ( Ac - Ab ) ] \times 100$$

As: Absorbance of specimen (well containing cells, test substance and Cell Counting Kit-8)
Ac: Absorbance of negative control (well containing cells, saline, and Cell Counting Kit-8)
Ab: Blank absorbance (well containing medium and Cell Counting Kit-8)

### <Results and Discussion>

In the drug sensitivity test, the dilution series of iABC2024K as the test substance was added to the 96-well plates (plate.1, plate.2, and plate. 3) in which BT-474 cells were seeded at 1.00×10⁴ cells/well, and the plates were subjected to culture for 3 days, 5 days, or 7 days. Then, Cell Counting Kit-8 was added, and the cell viability was measured 1 hour, 2 hours, and 4 hours after color development.

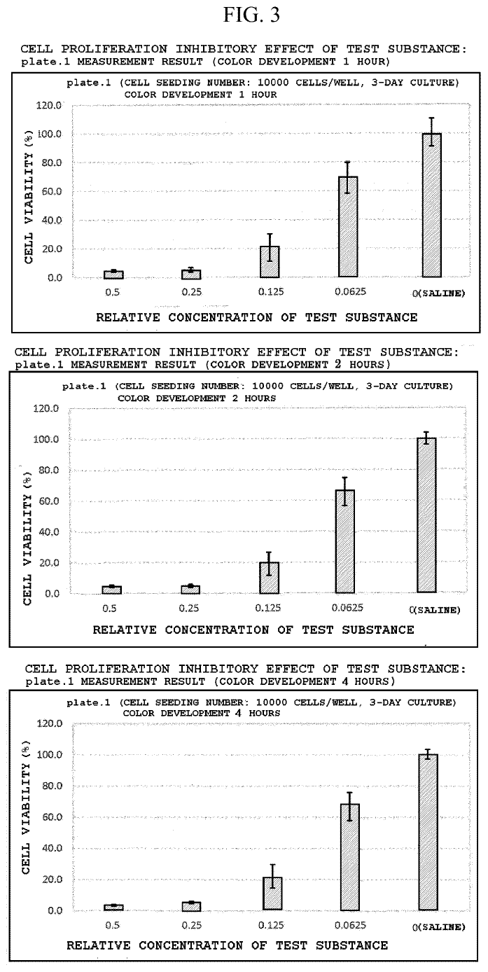

The results of plate.1 in which cells were seeded at 1.00×10⁴ cells/well and the cell viability was measured 3 days after the addition of the test substance are shown in FIG. 3.

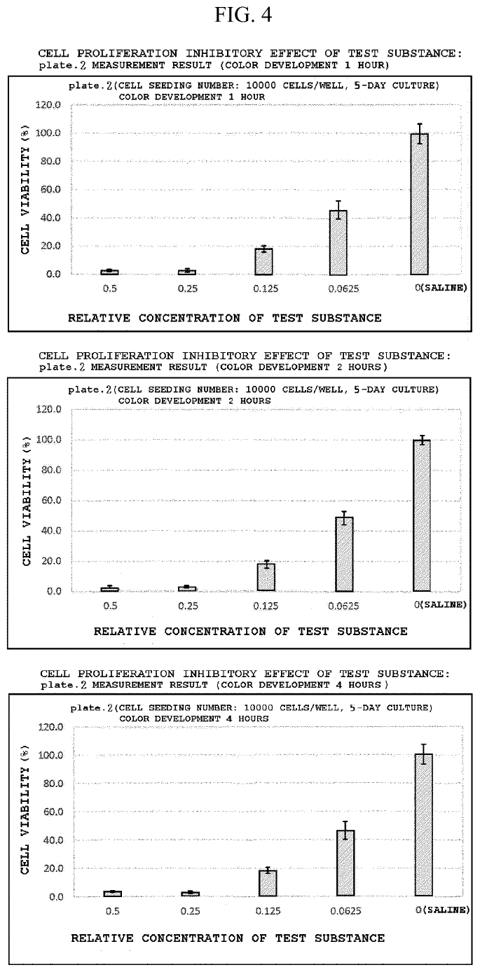

The results of plate.2 in which cells were seeded at 1.00×10⁴ cells/well and the cell viability was measured 5 days after the addition of the test substance are shown in FIG. 4.

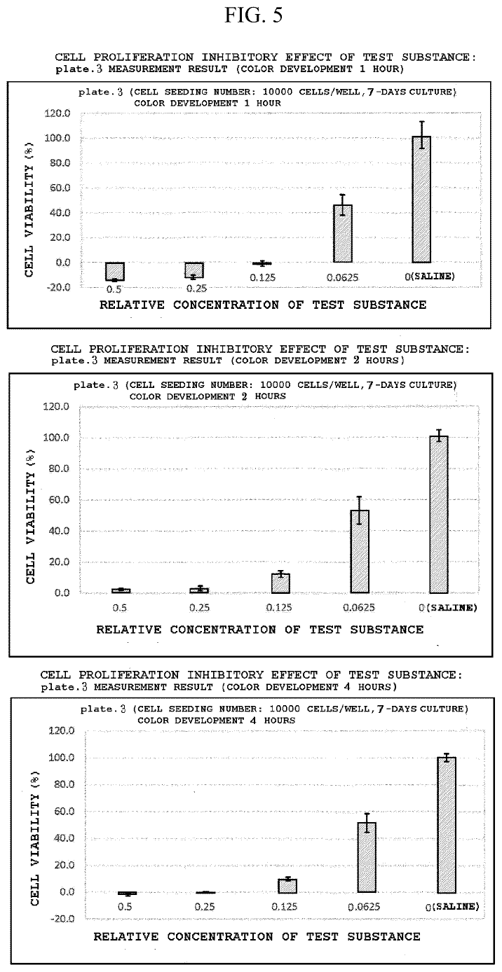

The results of plate. 3 in which cells were seeded at 1.00×10⁴ cells/well and the cell viability was measured 7 days after the addition of the test substance are shown in FIG. 5.

As a result, iABC2024K exhibited a concentration-dependent cell proliferation inhibitory effect over the entire concentration range (relative concentration: 0.0625 to 0.5) evaluated. Since iABC2024K is an intramuscular injection drug containing zapomeran developed as an mRNA vaccine as an active ingredient, it has been revealed that zapomeran exhibits a particularly remarkable cell proliferation inhibitory effect on breast cancer cells.

Although the details of the action mechanism of the cell proliferation inhibitory effect have not been clarified, in the evaluation test for the cell proliferation inhibitory effect described above, T cells, B cells, and the like involved in immunity are not present, and thus it is clear that the effect is based on at least an action mechanism other than the immune function.

Note that iABC2024K contains various additives in addition to the component of mRNA. However, these additives are, for example, a lipid nanoparticle for encapsulating mRNA, a simple emulsion stabilizer, a surfactant, a sweetener, a pH buffering agent, and the like, and it is not at all conceivable that these additives act on protein synthesis required for proliferation of breast cancer cells.

The proliferation inhibitor of breast cancer cells of the present invention can be suitably used for research of breast cancer cells and for treatment of breast cancer patients.

What is claimed is:

1. A proliferation inhibitor of breast cancer cells, comprising
an RNA molecule of a nucleotide sequence set forth in SEQ ID NO: 1, wherein
the RNA molecule includes a 5′ cap.

2. The proliferation inhibitor of breast cancer cells according to claim 1, wherein the 5′ cap has the following chemical structure:

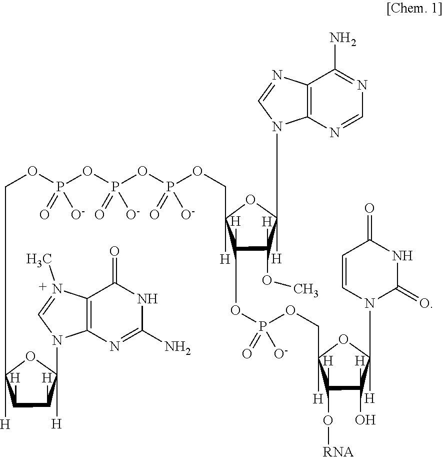

3. The proliferation inhibitor of breast cancer cells according to claim 1, wherein the RNA molecule is encapsulated in a lipid nanoparticle (LNP).

4. A therapeutic agent for breast cancer, comprising
an RNA molecule of a nucleotide sequence set forth in SEQ ID NO: 1, wherein
the RNA molecule includes a 5′ cap.

5. The therapeutic agent for breast cancer according to claim 4, wherein the 5′ cap has the following chemical structure:

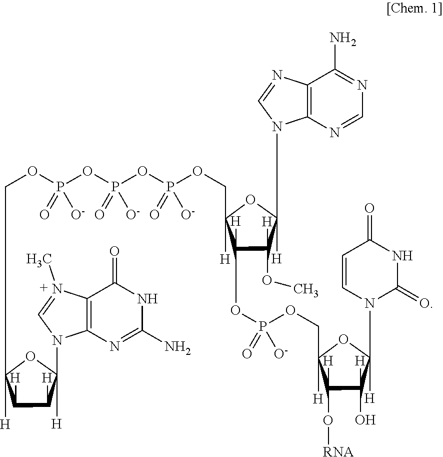

6. The therapeutic agent for breast cancer according to claim 4, wherein the RNA molecule is encapsulated in a lipid nanoparticle (LNP).


SEQUENCE LISTING (1 sequence)

SEQ ID NO: 1 - RNA, 11861 nt, synthetic construct
  source 1..11861: mol_type = other RNA
gatgggcggcgcatgagagaagcccagaccaattacctacccaaaatggagaaagttcacgttgacatcgaggaagacagcccattcctcagagccttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaatcggacaagaaaatgaaggagctggccgccgtcatgagcgaccctgacctggaaactgagactatgtgcctccacgacgacgagtcgtgtcgctacgaagggcaagtcgctgtttaccaggatgtatacgccgtcgacggccccaccagcctgtaccaccaggccaacaagggcgtgagggtggcctactggatcggcttcgacaccacacccttcatgttcaagaacctggccggcgcctaccccagctacagcaccaactgggccgacgagacagtgctgaccgccaggaacatcggcctgtgcagcagcgacgtgatggagaggagccggaggggcatgagcatcctgaggaagaagtacctgaagcccagcaacaacgtgctgttcagcgtgggcagcaccatctaccacgagaagagggacctgctgaggagctggcacctgcccagcgtgttccacctgaggggcaagcagaactacacctgcaggtgcgagacaatcgtgagctgcgacggctacgtggtgaagaggatcgccatcagccccggcctgtacggcaagcccagcggctacgccgccaccatgcacagggagggcttcctgtgctgcaaggtgaccgacaccctgaacggcgagagggtgagcttccccgtgtgcacctacgtgcccgccaccctgtgcgaccagatgaccggcatcctggccaccgacgtgagcgccgacgacgcccagaagctgctggtgggcctgaaccagaggatcgtggtgaacggcaggacccagaggaacaccaacaccatgaagaactacctgctgcccgtggtggcccaggccttcgccaggtgggccaaggagtacaaggaggaccaggaggacgagaggcccctgggcctgagggaccgacagctggtgatgggctgctgctgggccttcaggcggcacaagatcaccagcatctacaagaggcccgacacccagaccatcatcaaggtgaacagcgacttccacagcttcgtgctgcccaggatcggcagcaacaccctggagatcggcctgaggacccggatcaggaagatgctggaggagcacaaggagcccagccctctgatcaccgccgaggacgtgcaggaggccaagtgcgccgccgacgaggccaaggaggtgagggaggccgaggagctgagggccgccctgcctcccctggccgccgacgtggaggagcccaccctggaggccgacgtggacctgatgctgcaggaggccggcgccggcagcgtggagacacccaggggcctgatcaaggtgaccagctacgacggcgaggacaagatcggcagctacgccgtgctcagccctcaggccgtgctgaagtccgagaagctgagctgcatccaccctctggccgagcaggtgatcgtgatcacccacagcggcaggaagggcaggtacgccgtggagccctaccacggcaaggtggtggtccccgagggccacgccatccccgtgcaggacttccaggccctgagcgagagcgccaccatcgtgtataacgagagggagttcgtgaacaggtacctgcaccacatcgccacccacggcggcgccctgaacaccgacgaggagtactacaagaccgtgaagcccagcgagcacgacggcgagtacctgtacgacatcgacaggaagcagtgcgtgaagaaggagctggtgaccggcctgggcctgaccggcgagctggtggaccctcccttccacgagttcgcctacgagagcctgaggaccaggcccgccgctccctaccaggtgcccaccatcggcgtgtacggcgtgcccggcagcggcaagagcggcatcatcaagagcgccgtgaccaagaaggacctggtggtgagcgccaagaaggagaactgcgccgagatcatcagggacgtgaagaagatgaagggcctggacgtgaacgccaggaccgtggacagcgtgctcctgaacggctgcaagcaccccgtggagacactgtatatcgacgaggccttcgcctgccacgccggcaccctgagggccctgatcgccatcatcaggcccaagaaggccgtgctgtgcggcgaccccaagcagtgcggcttcttcaacatgatgtgcctgaaggtgcacttcaaccacgagatctgcacccaggtgttccacaagagcatcagcaggcggtgcaccaagagcgtgaccagcgtggtgagcaccctgttctacgacaagaagatgaggaccaccaaccccaaggagacaaagatcgtgatcgacaccaccggcagcaccaagcccaagcaggacgacctgatcctgacctgcttcaggggctgggtgaagcagctgcagatcgactacaagggcaacgagatcatgaccgccgccgctagccagggcctgaccaggaagggcgtgtacgccgtgaggtacaaggtgaacgagaatcccctgtacgcccctaccagcgagcacgtgaacgtcctgctgaccaggaccgaggacaggatcgtgtggaagaccctggccggcgacccctggatcaagaccctgaccgccaagtaccccggcaacttcaccgccaccatcgaggagtggcaggccgagcacgacgccatcatgaggcacatcctggagaggcccgaccccaccgacgtgttccagaacaaggccaacgtgtgctgggccaaggccctggtgcccgtgctgaagaccgccggcatcgacatgaccaccgagcagtggaacaccgtggactacttcgagacagacaaggcccacagcgccgagatcgtgctgaaccagctgtgcgtgaggttcttcggcctggacctggacagcggcctgttcagcgcccctaccgtgcccctgagcatcaggaacaaccactgggacaacagccccagccccaacatgtacggcctgaacaaggaggtggtgaggcagctgagcaggcggtaccctcagctgcccagggccgtggccaccggcagggtgtacgacatgaacaccggcaccctgaggaactacgaccccaggatcaacctggtgcccgtgaacaggcggctgccacacgccctggtgctgcaccacaacgagcaccctcagagcgacttcagcagcttcgtgagcaagctgaagggcaggaccgtgctggtggtgggcgagaagctgagcgtgcccggcaagatggtggactggctgagcgacaggcccgaggccaccttccgggccaggctggacctgggcatccccggcgacgtgcccaagtacgacatcatcttcgtgaacgtgaggaccccttacaagtaccaccactaccagcagtgcgaggaccacgccatcaagctgagcatgctgaccaagaaggcctgcctgcacctgaaccccggcggcacctgcgtgagcatcggctacggctacgccgacagggccagcgagagcatcatcggcgccatcgccaggctgttcaagttcagcagggtgtgcaagcccaagagcagcctggaggagacagaggtgctgttcgtgttcatcggctacgaccggaaggccaggacccacaacccctacaagctgagcagcaccctgaccaacatctacaccggcagcaggctgcacgaggccggctgcgcccctagctaccacgtggtgaggggcgacatcgccaccgccaccgagggcgtgatcatcaacgccgccaacagcaagggccagcccggcggcggggtgtgcggcgccctgtataagaagttccccgagagcttcgacctgcagcccatcgaggtgggcaaggccaggctggtgaagggcgccgccaagcacatcatccacgccgtgggccccaacttcaacaaggtgagcgaggtggagggcgacaagcagctggccgaggcctacgagagcatcgccaagatcgtgaacgacaacaactacaagagcgtggccatccctctgctgagcaccggcatcttcagcggcaacaaggacaggctgacccagagcctgaaccacctgctgaccgccctggacaccaccgacgccgacgtggccatctactgcagggacaagaagtgggagatgaccctgaaggaggccgtggccaggcgggaggccgtggaggagatctgcatcagcgacgacagcagcgtgacggagcccgacgccgagctggtgagggtgcaccccaagagcagcctggccggcaggaagggctacagcaccagcgacggcaagaccttcagctacctggagggcaccaagttccaccaggccgccaaggacatcgccgagatcaacgccatgtggcccgtggccaccgaggccaacgagcaggtgtgcatgtatatcctgggcgagagcatgagcagcatcaggagcaagtgccccgtggaggagagcgaggccagcacccctcccagcaccctgccctgcctgtgcatccacgccatgacccctgagagggtgcagcggctgaaggccagcaggcccgagcagatcaccgtgtgcagcagcttccctctgcccaagtaccggatcaccggcgtgcagaagatccagtgcagccagcccatcctgttcagccccaaggtgcccgcctacatccaccccaggaagtacctggtggagacaccccccgtggacgagacacccgagcccagcgccgagaaccagagcaccgagggcacccctgagcagcctcccctgatcaccgaggacgagacaaggaccaggacgcccgagcccatcatcattgaggaggaagaggaggacagcatcagcctgctgagcgacggccccacccaccaggtgctgcaggtggaggccgacatccacggccctcccagcgtgagcagctccagctggagcatccctcacgccagcgacttcgacgtggacagcctgagcatcctggacaccctggagggcgccagcgtgaccagcggcgccaccagcgccgagacaaacagctacttcgccaagagcatggagttcctggccaggcccgtgcccgcccctaggaccgtgttcaggaaccctccccaccccgcccctaggaccaggacccctagcctggcccctagcagggcctgcagcaggaccagcctggtgagcacccctcccggcgtgaaccgggtgatcaccagggaggagctggaggccctgacccctagcaggacccctagcaggagcgtgagcaggaccagcctggtgagcaaccctcccggcgtgaaccgggtgatcaccagggaggagttcgaggccttcgtggcccagcagcaaaggcggttcgacgccggcgcctacatcttcagcagcgacaccggccagggccacctgcagcagaagtccgtgaggcagaccgtgctgagcgaggtggtcctggagaggacggagctggagatcagctacgcccctaggctggaccaggagaaggaggagctgctgaggaagaagctgcagctgaaccccacccctgccaacaggagcaggtaccagagcaggaaggtggagaacatgaaggccatcaccgccaggcggatcctgcagggcctgggccactacctgaaggccgagggcaaggtggagtgctacaggaccctgcaccccgtgcccctgtactccagctccgtgaacagggccttcagcagccccaaggtggccgtggaggcctgcaacgccatgctgaaggagaacttccccaccgtggccagctactgcatcatccccgagtacgacgcctacctggacatggtggacggcgccagctgctgcctggacaccgccagcttctgccccgccaagctgaggagcttccccaagaagcacagctacctggagcccaccatcaggagcgccgtgcccagcgccatccagaacaccctgcagaacgtgctggccgccgctaccaagaggaactgcaacgtgacccagatgagggagctgcccgtgctggacagcgccgccttcaacgtggagtgcttcaagaagtacgcctgcaacaacgagtactgggagacattcaaggagaaccccatcaggctgaccgaggagaacgtggtgaactacatcaccaagctgaagggccccaaggccgccgctctgttcgccaagacccacaacctgaacatgctccaggacatccctatggacaggttcgtgatggacctgaagagggacgtgaaggtgacccctggcaccaagcacaccgaggagaggcccaaggtgcaggtgatccaggccgccgaccctctggccaccgcctacctgtgcggcatccacagggagctggtgaggcggctgaacgccgtcctgctgcccaacatccacaccctgttcgacatgagcgccgaggacttcgacgccatcatcgccgagcacttccagcccggcgactgcgtgctggagacagacatcgccagcttcgacaagagcgaggacgacgctatggccctgaccgccctgatgatcctggaggacctgggcgtggacgccgagctgctgaccctgatcgaggccgccttcggcgagatcagcagcatccacctgcccaccaagaccaagttcaagttcggcgccatgatgaagtccggcatgttcctgaccctgttcgtgaacaccgtgatcaacatcgtgatcgccagcagggtgctgcgggagaggctgaccggcagcccctgcgccgccttcatcggcgacgacaacatcgtgaagggcgtgaagtccgacaagctgatggccgacaggtgcgccacctggctgaacatggaggtgaagatcatcgacgccgtggtgggcgagaaggccccttacttctgcggcggcttcatcctgtgcgacagcgtgaccggcaccgcctgcagggtggccgaccctctgaagaggctgttcaagctgggcaagcccctggccgccgacgacgagcacgacgacgataggcggagggccctgcacgaggagagcaccaggtggaaccgggtgggcatcctgagcgagctgtgcaaggccgtggagagcaggtacgagacagtgggcaccagcatcatcgtgatggccatgaccaccctggccagcagcgtcaagtccttcagctacctgaggggggcccctataactctctacggctaacctgaatggactacgacatagtctagtccgccaaggccgccaccatgttcgtgttcctggtgctgctgcccctggtgtctagccagtgcgtgaacctgaccaccaggacccagctgcctcccgcctacaccaacagcttcaccaggggcgtgtactaccccgacaaggtgttcaggagcagcgtgctgcacagcacccaggacctgttcctgcccttcttcagcaacgtgacctggttccacgccatccacgtgagcggcaccaacggcaccaagaggttcgacaaccccgtgctgcccttcaacgacggcgtgtacttcgccagcaccgagaagtccaacatcatcaggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagggtgtactccagcgccaacaactgcaccttcgagtacgtgagccagcccttcctgatggacctggagggcaagcagggcaacttcaagaacctgagggagttcgtgttcaagaacatcgacggctacttcaagatctacagcaagcacacccctatcaacctggtgagggacctgccccagggcttcagcgccctggagcccctggtggacctgcccatcggcatcaacatcaccaggttccagaccctgctggccctgcacaggagctacctgacccctggcgacagcagctccggctggaccgccggcgccgccgcttactacgtgggctacctgcagcccaggaccttcctgctgaagtacaacgagaacggcaccatcaccgacgccgtggactgcgccctggaccctctgagcgagacaaagtgcaccctgaagtccttcaccgtggagaagggcatctaccagaccagcaacttcagggcgcagcccaccgagagcatcgtgaggttccccaacatcaccaacctgtgccccttcggcgaggtgttcaacgccaccaggttcgccagcgtgtacgcctggaacaggaagaggatcagcaactgcgtggccgactacagcgtgctgtataacagcgccagcttcagcaccttcaagtgctacggcgtgagccccaccaagctgaacgacctgtgcttcaccaacgtgtacgccgacagcttcgtgatcaggggcgacgaggtgaggcagatcgcccctggccagaccggcaagatcgccgactacaactacaagctgcccgacgacttcaccggctgcgtgatcgcctggaacagcaacaacctggacagcaaggtgggcggcaactacaactacctgtaccggctgttcagaaagagcaacctgaagcccttcgagagggacatcagcaccgagatctaccaggccggcagcaccccttgcaacggcgtggagggcttcaactgctacttccctctgcagagctacggcttccagcccaccaacggcgtgggctaccagccctacagggtggtggtcctgagcttcgagctgctgcacgcccctgccaccgtgtgcggccccaagaagtccaccaacctggtgaagaacaagtgcgtgaacttcaacttcaacggcctgaccggcaccggcgtgctgaccgagagcaacaagaagttcctgcccttccagcagttcggcagggacatcgccgacaccaccgacgccgtgagggaccctcagaccctggagatcctggacatcaccccttgcagcttcggcggcgtgagcgtgatcacccctggcaccaacaccagcaaccaggtggccgtgctgtaccagggcgcgaactgcaccgaggtgcccgtggccatccacgccgaccagctgacccctacctggagggtgtactccaccggcagcaacgtgttccagaccagggccggctgcctgatcggcgccgagcacgtgaacaacagctacgagtgcgacatccccatcggcgccggcatctgcgccagctaccagacccagaccaacagccccgggagcgccagcagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtatatctgcggcgacagcaccgagtgcagcaacctgctgccccagtacggcagcttctgcacccagccgaacagggccctgaccggcatcgccgtggagcaggacaagaacacccaggaggtgttcgcccaggtgaagcagatctacaagacccctcccatcaaggacttcggcggcttcaacttcagccagatcctgcccgaccccagcaagcccagcaagaggagcttcatcgaggacctgctgttcaacaaggtgaccctggccgacgccggcttcatcaagcagtacggcgactgcctgggcgacatcgccgccagggacctgatctgcgcccagaagttcaacggcctgaccgtgctgcctcccctgctgaccgacgagatgatcgcccagtacaccagcgccctgctggccggcaccatcaccagcggctggaccttcggcgccggcgccgccctgcagatccccttcgccatgcagatggcctacaggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcaggctggacccacccgaggccgaggtgcagatcgacaggctgatcaccggcaggctgcagagcctgcagacctacgtgacccagcagctgatcagggccgccgagatcagggccagcgccaacctggccgccaccaagatgagcgagtgcgtgctgggccagagcaagagggtggacttctgcggcaagggctaccacctgatgagcttccctcagagcgcccctcacggcgtggtgttcctgcacgtgacctacgtgcccgcccaggagaagaacttcaccacagcccctgccatctgccacgacggcaaggcccacttccccagggagggcgtgttcgtgagcaacggcacccactggttcgtgacccagaggaacttctacgagccccagatcatcaccaccgacaacaccttcgtgagcggcaactgcgacgtggtgatcggcatcgtgaacaacaccgtgtacgaccctctgcagcccgagctggacagcttcaaggaggagctggacaagtacttcaagaaccacaccagccccgacgtggacctgggcgacatcagcggcatcaacgccagcgtggtgaacatccagaaggagatcgacaggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctaaactcgagtatgttacgtgcaaaggtgattgtcaccccccgaaagaccatattgtgacacaccctcagtatcacgcccaaacatttacagccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa